(19) United States
(12) Patent Application Publication
(10) Pub. No.: US 2023/0181235 A1
(43) Pub. Date: Jun. 15, 2023

(54) ELECTRODE ASSEMBLY

(71) Applicant: GYRUS MEDICAL LIMITED, Cardiff (GB)

(72) Inventors: James DICKSON, Cardiff (GB); Sunny RUPALIA, Cardiff (GB); Darren YOUNG, Cardiff (GB)

(73) Assignee: GYRUS MEDICAL LIMITED, Cardiff (GB)

(21) Appl. No.: 18/079,429

(22) Filed: Dec. 12, 2022

Related U.S. Application Data
(60) Provisional application No. 63/289,909, filed on Dec. 15, 2021.

(51) Int. Cl. A61B 18/14

(52) U.S. Cl. CPC A61B 18/14; A61B 2017/00526

(57) ABSTRACT

An electrode assembly for an electrosurgical instrument includes an active electrode and retainer arrangement that are inserted to a cavity of an insulator and then welded together to secure the active electrode in the insulator for use in tissue treatment. The active electrode and retainer are configured to interlock with at least one internal retention surface of the cavity. Once assembled in the cavity, both the active electrode and the retainer may have a portion that extends out of the cavity to protrude from the insulator. This allows opposing biasing forces to be exerted on the active electrode and the retainer respectively so as to push the two components against a retention surface within the cavity. When the active electrode and the retainer are welded together, any clearance with the retention surfaces inside the cavity, caused by variations between parts during the manufacturing process, is removed or at least minimized.

### TECHNICAL FIELD

The present invention relates to electrosurgical instruments. More specifically, the present invention relates to an electrode assembly for an electrosurgical instrument and a method of manufacture.

### BACKGROUND TO THE INVENTION AND PRIOR ART

Surgical instruments, including radio frequency (RF) electrosurgical instruments, have become widely used in surgical procedures where access to the surgical site is restricted to a narrow passage, for example, in minimally invasive “keyhole” surgeries.

RF electrosurgical instruments may include an active electrode which forms a distal RF tip of the instrument, and a return electrode. The distal RF tip provides tissue ablation and/or coagulation effects at a surgical site when a RF power signal is delivered to the electrodes. Typical RF electrosurgical instruments also often use a saline suction pathway at the distal tip during either ablating or coagulating tissue.

Known wet-field RF hand instruments, for applications such as arthroscopy, have an RF tip that is both mechanically and electrically connected to the rest of the instrument assembly via various means. Typically, this is through a mechanical interlock that doubles as an electrical connection, or via a mechanical interlock/adhesive with a secondary connection to an active RF wire, to transmit RF energy.

FIG. 2A illustrates an example of a distal RF tip 20 in a prior art device. In such arrangements, there is an ample space envelope to create a large and robust mechanical interlocking feature or apply adhesive. The region where the RF ablation and erosion will occur is labelled 26, whilst region containing the interlocking feature to which adhesive will be applied is labelled 28. In the distal tip 20 shown in FIG. 2A, the interlocking feature is created with a tube 22 that is slotted over a portion 24 of the active tip, and then welded in place. Whilst being relatively mechanically robust, this interlocking feature takes up a large amount of space in the suction pathway.

Some RF instruments also implement a mechanical shaver, with the main mechanical shaving componentry located on the opposite side of the distal tip. An RF shaver distal tip 30 is illustrated by FIG. 2B, wherein the distal tip 30 comprises an active RF tip 32 and a rotating inner blade 34 disposed within a ceramic insulator 36. As such, there is no space to fit an interlocking feature such as that shown in FIG. 2A due to the rotating inner blade 34, such that the region available for providing mechanical retention (labelled 38) is significantly reduced. Therefore, a different method is required to retain the active RF tip 32 mechanically within the ceramic insulator 36.

One previous solution, as described in GB publication number 2590929, provides a tip retention mechanism arranged to fit into a low depth profile within the ceramic insulator. This solution provides an insulator with a single inter-locking angled plane and two-part electrode. Such an arrangement is reliant upon achieving optimum tolerances between the mating insulator, electrode and retainer components, however in practice, once manufacturing tolerances and clearances for assembly are applied, the amount of remaining interlock can be minimal. Furthermore, the amount of ceramic insulator in some cases may not be mechanically robust enough to withstand the expected clinical loads. Similarly, the area of the RF active tip for delivering a tissue effect is relatively large and has an exposed ceramic insulator portion in the centre that may negatively impact on the performance of the ablation and coagulation functions.

There is thus a need to further improve the retention of the active electrode within such RF electrosurgical instruments.

### SUMMARY OF THE INVENTION

In known RF electrosurgical instruments that implement an active electrode tip and a mechanical shaver, the space available for providing secure retention of the active electrode tip is significantly reduced. The present disclosure therefore seeks to address this problem by providing an active electrode and retainer arrangement that are inserted to a cavity of an insulator and then welded together to secure the active electrode in the insulator for use in tissue treatment. The active electrode and retainer are configured to interlock with at least one internal retention surface of the cavity. Once assembled in the cavity, both the active electrode and the retainer may have a portion that extends out of the cavity, such that it protrudes from the insulator. This allows opposing biasing forces to be exerted on the active electrode and the retainer respectively so as to push the two components against a retention surface within the cavity. Consequently, when the active electrode and the retainer are welded together, any clearance with the retention surfaces inside the cavity, for example, caused by variations between parts during the manufacturing process, is removed or at least minimised. As such, the amount of interlock with the retention surfaces of the cavity can be maximised, thus providing a more robust retention of the active electrode. Furthermore, as the interlocking parts of the insulator are provided beneath the main body of the electrode, the overall active tip area is reduced, which thereby improves the performance of the ablation and coagulation functions.

A first aspect of the present invention provides a method of manufacturing an electrode assembly for use in an end effector of an electrosurgical instrument, comprising providing an outer casing comprising an insulating material, wherein the outer casing comprises a cavity, the cavity comprising a base surface, and a first projection extending from the base surface, the first projection comprising a first retention surface and a second retention surface, wherein at least one of the first and second retention surfaces extends away from the base surface at a non-transverse angle relative to a longitudinal axis of the outer casing, inserting a retainer into the cavity via a first opening in a first wall of the outer casing, wherein the retainer comprises a first retention feature configured to engage the first retention surface, inserting an electrode into the cavity via the first opening, wherein the electrode comprises a second retention feature configured to engage the second retention surface, and joining the electrode and the retainer to form the electrode assembly, such that the electrode assembly is mechanically retained within the outer casing.

In some arrangements, the method further comprises applying a first biasing force on the retainer such that first retention feature is biased against the first retention surface, and a second biasing force on the electrode such that the second retention feature is biased against the second retention surface.

By biasing the electrode and the retainer such that they are biased against the respective retention surfaces so as to remove any clearance before the two components are joined together. In doing so, any part-to-part variations introduced during the manufacturing process are mitigated, thereby maximising the amount of interlock with the retention surfaces.

The biasing forces may be applied by any suitable means. For example, the first and second biasing forces may be applied by placing a magnet in the vicinity of the electrode and the retainer. More specifically, the magnet may be placed within a central location of the electrode assembly such as a suction hole so as to pull the electrode and retainer inwards against the respective retention surfaces.

In other arrangements, the retainer may further comprise a protrusion configured to extend out of the first opening such that the protrusion extends beyond the first wall. The first biasing force may then be applied to the protrusion. Similarly, at least one portion of the electrode may be configured to extend out of the first opening such that the at least one portion extends beyond the first wall. In such cases, the second biasing force may be applied to the at least one portion. As such, both the electrode and the retainer may comprise features that extend beyond the profile of the outer casing to allow biasing forces to be applied. For example, the two biasing forces may be applied to the electrode and the retainer using a mechanical jig, or some other suitable apparatus.

In some arrangements, the first retention surface may extend away from the base surface at an angle transverse to the longitudinal axis, and the second retention surface may extend away from the base surface in a direction towards the distal end of the outer casing at a non-transverse angle relative to a longitudinal axis. That is to say, the first retention surface may be a vertical surface, whilst the second retention surface may be an angled surface.

In other arrangements, the first retention surface may extend away from the base surface in a direction towards the proximal end of the outer casing at a non-transverse angle relative to a longitudinal axis, and the second retention surface may extend away from the base surface in a direction towards the distal end of the outer casing at a non-transverse angle relative to a longitudinal axis. That is to say, both of the retention surfaces may be angled surfaces, with one extending towards the distal end of the outer casing and the other extending in the opposite direction. By providing two angled retention surfaces, the strength of the mechanical retention of the electrode and the retainer is further increased.

In other arrangements, the first retention surface may extend away from the base surface in a first lateral direction at a non-transverse angle relative to a longitudinal axis, and the second retention surface may extend away from the base surface in a second lateral direction at a non-transverse angle relative to a longitudinal axis. That is to say, the retention surfaces may be angled surfaces that extend towards opposing sides of the outer casing. One advantage of this arrangement is that the interlocking mechanism is moved out of the proximal end of the cavity, which typically comprises an entry port for receiving an electrical conductor for connecting the electrode assembly to an electrical supply. Consequently, the proximal inner wall can be made vertical, which vastly reduces the likelihood of excess material building up around the entry port during moulding, with the conductor also then providing a third retention point once it has been assembled and connected to the electrode assembly. As such, a robust mechanical retention that is easy to manufacture is provided.

Preferably, the electrode comprises a tissue treatment surface, the tissue treatment surface being configured to cover the first projection. By providing an arrangement where the interlocking mechanism (i.e. the first projection and the retention features) are provided below the treatment surface, the overall active electrode area is reduced, and the overall performance of the ablation and coagulation functions is improved.

The first projection may comprise a first suction aperture, and the tissue treatment surface may comprise a second suction aperture, the electrode being inserted to the cavity such that the second suction aperture aligns with first suction aperture.

The first projection may be connected to an internal wall of the cavity via an interconnecting wall. That is to say, the first projection may be connected to the outer perimeter of the outer casing to further increase the mechanical strength of the first projection.

The retainer may comprise an electrically conductive material.

The insulating material may comprise a ceramic material.

Joining the electrode and the retainer may comprise welding the electrode and the retainer.

The method may further comprise providing a conductor, the conductor being received within the cavity via a second opening in an internal wall, wherein the conductor is in electrical communication with the electrode assembly.

A second aspect of the present invention provides an electrode assembly for use in an end effector of an electrosurgical instrument, the electrode assembly comprising an electrode, a retainer, and an outer casing comprising an insulating material, wherein the outer casing comprises a cavity configured to receive the electrode and the retainer, the cavity comprising a base surface, and a first projection extending from the base surface, the first projection comprising a first retention surface and a second retention surface, wherein at least one of the first and second retention surfaces extends away from the base surface at a non-transverse angle relative to a longitudinal axis of the outer casing, characterised in that the electrode and the retainer are provided in the outer casing by inserting a retainer into the cavity via a first opening in a first wall of the outer casing, wherein the retainer comprises a first retention feature configured to engage the first retention surface, inserting the electrode into the cavity via the first opening, wherein the electrode comprises a second retention feature configured to engage the second retention surface, and joining the electrode and the retainer to form the electrode assembly, such that the electrode assembly are mechanically retained within the outer casing.

In some arrangements, a first biasing force may be applied to the retainer such that first retention feature is biased against the first retention surface, and a second biasing force may be applied to the electrode such that the second retention feature is based against the second retention surface.

A third aspect of the present invention provides an electrode assembly for use in an end effector of an electrosurgical instrument, the electrode assembly comprising an outer casing comprising an insulating material, wherein the outer casing comprises a cavity, the cavity comprising a base surface, and a first projection extending from the base surface, the first projection comprising a first retention surface and a second retention surface, wherein the first and second retention surfaces extend away from the base surface at a non-transverse angle relative to a longitudinal axis of the outer casing, and an electrode arrangement received in the cavity, the electrode arrangement comprising a retainer and an electrode in electrical communication with the retainer, wherein the retainer comprises a first retention feature configured to engage the first retention surface, and the electrode comprises a second retention feature configured to engage the second retention surface, such that electrode arrangement is mechanically retained within the outer casing.

In some arrangements, the first retention surface may extend away from the base surface in a direction towards the proximal end of the outer casing at a non-transverse angle relative to a longitudinal axis, and the second retention surface may extend away from the base surface in a direction towards the distal end of the outer casing at a non-transverse angle relative to a longitudinal axis.

In other arrangements, the first retention surface may extend away from the base surface in a first lateral direction at a non-transverse angle relative to a longitudinal axis, and the second retention surface may extend away from the base surface in a second lateral direction at a non-transverse angle relative to a longitudinal axis.

The retainer and the electrode may be welded together within the cavity.

Another aspect of the present invention provides an electrosurgical instrument, comprising a hand-piece, one or more user-operable buttons on the handpiece that control the instrument to operate, and an operative shaft, having RF electrical connections, and drive componentry for an end effector, the electrosurgical instrument further comprising an end effector having an electrode assembly in accordance with the aspects described above, the electrode being connected to the RF electrical connections.

The end effector may also comprise a rotary shaver arrangement being operably connected to the drive componentry to drive the rotary shaver to operate in use.

A further aspect of the present invention provides an electrosurgical system, comprising an RF electrosurgical generator, a suction source, and an electrosurgical instrument as described above, the arrangement being such that in use the RF electrosurgical generator supplies an RF coagulation or ablation signal via the RF electrical connections to the active electrode.

Yet a further aspect of the present invention may provide a method for processing an instrument for surgery, the method comprising obtaining an electrosurgical instrument and/or end effector described herein, sterilizing the electrosurgical instrument and/or end effector, and storing the electrosurgical instrument and/or end effector in a sterile container.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 shows an example of the electrosurgical instrument system comprising an RF electrosurgical instrument according to the present invention;

FIGS. 2A-B illustrate part of an end effector known in the prior art;

FIGS. 3A-C illustrate a first example of an end effector in accordance with the present invention;

FIGS. 4A-C illustrate a second example of an end effector in accordance with the present invention;

FIGS. 5A-D illustrate a third example of an end effector in accordance with the present invention.

### DETAILED DESCRIPTION OF THE EMBODIMENTS

In known RF electrosurgical instruments that implement an active electrode tip and a mechanical shaver, the space available for providing secure retention of the active electrode tip is significantly reduced. The present disclosure therefore seeks to address this problem by providing an active electrode and retainer arrangement that are inserted to a cavity on one side of an insulator such that they interlock with a feature of the insulator inside the cavity, biased together so as to maximise the degree of interlock and then welded together to secure the active electrode in the insulator for use in tissue treatment.

The active electrode and retainer are configured to interlock with at least one internal surface of the cavity. In this respect, the cavity of the insulator is provided with an interlocking projection defined by a first retention surface and a second retention surface extending from a base surface of the cavity. At least one of the retention surfaces extends from the base surface at a non-transverse angle to the longitudinal axis of the insulator, to thereby provide an undercut with which a part of the active electrode and/or retainer can interlock. Once assembled in the cavity, both the active electrode and the retainer have a portion that extends out of the cavity, that is to say, at least a portion of the active electrode and the retainer protrudes from the insulator. This allows opposing biasing forces to be exerted on the active electrode and the retainer respectively so as to push the two components against the respective retention surface within the cavity. That is to say, the active electrode is biased against one retention surface, and the retainer is biased against the other retention surface. The active electrode and retainer are then welded together, to retain the active electrode within the insulator.

By applying these biasing forces during welding, any clearance between the active electrode, the retainer, and the respective retention surfaces, for example, caused by variations between parts during the manufacturing process, is removed or at least minimised. As such, the amount of interlock with the retention surfaces can be maximised, thus providing a more robust retention of the active electrode. Furthermore, as the interlocking parts of the insulator are provided beneath the active electrode, the overall active tip area is reduced, which thereby improves the performance of the ablation and coagulation functions.

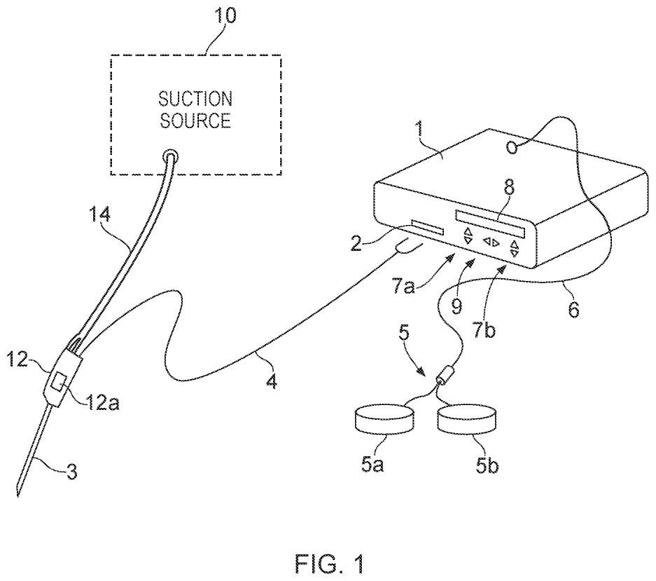

FIG. 1 shows an electrosurgical apparatus including an electrosurgical generator 1 having an output socket 2 that provides a radio frequency (RF) output (e.g. a RF power signal), via a connection cord 4, to an electrosurgical instrument 3. The instrument 3 has a suction tube 14 which is connected to a suction source 10. Activation of the generator 1 may be performed from the instrument 3 via a handswitch 12a on the handpiece 12 of the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode, or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The electrosurgical instrument 3 is a dual sided (or an opposite sided) RF shaver device. In this respect, the main RF componentry and the main mechanical shaving/cutting componentry of the instrument 3 can be provided on opposite sides of a distal end portion of the instrument 3. In such cases, any of the switching means described above may be provided for selecting between the RF ablation or coagulation mode, and the mechanical shaving mode. The structure of the distal end of the instrument 3 is described in more detail below.

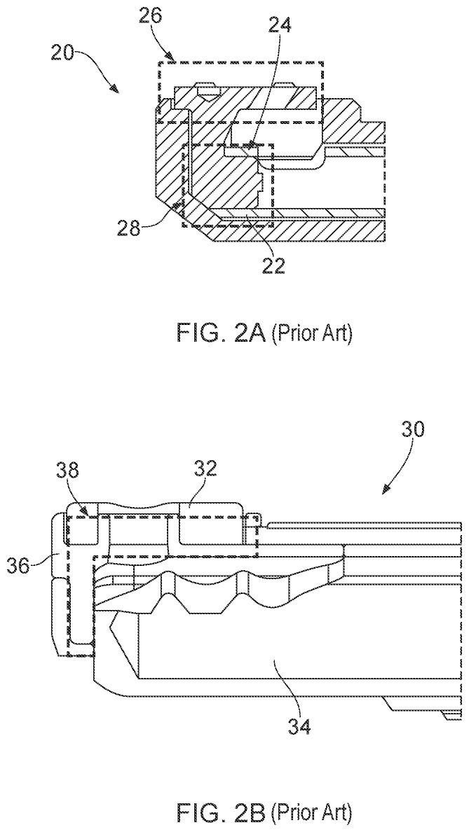

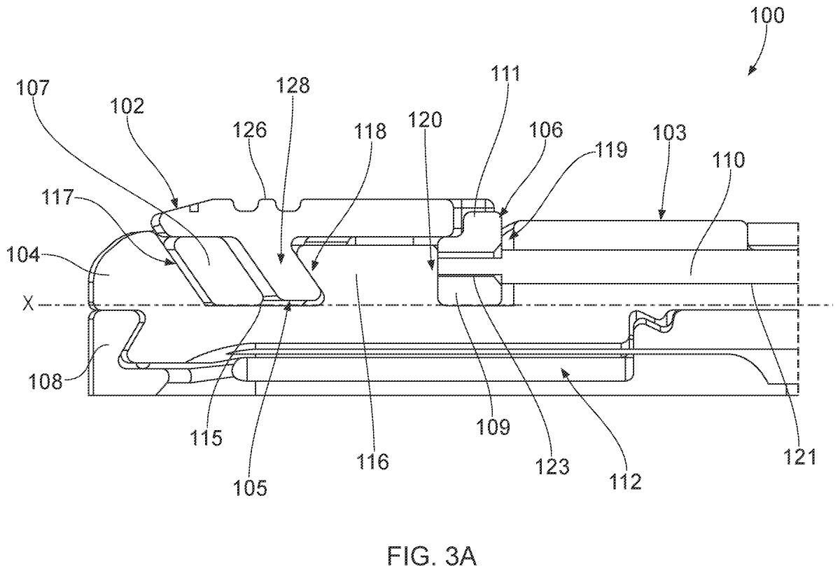

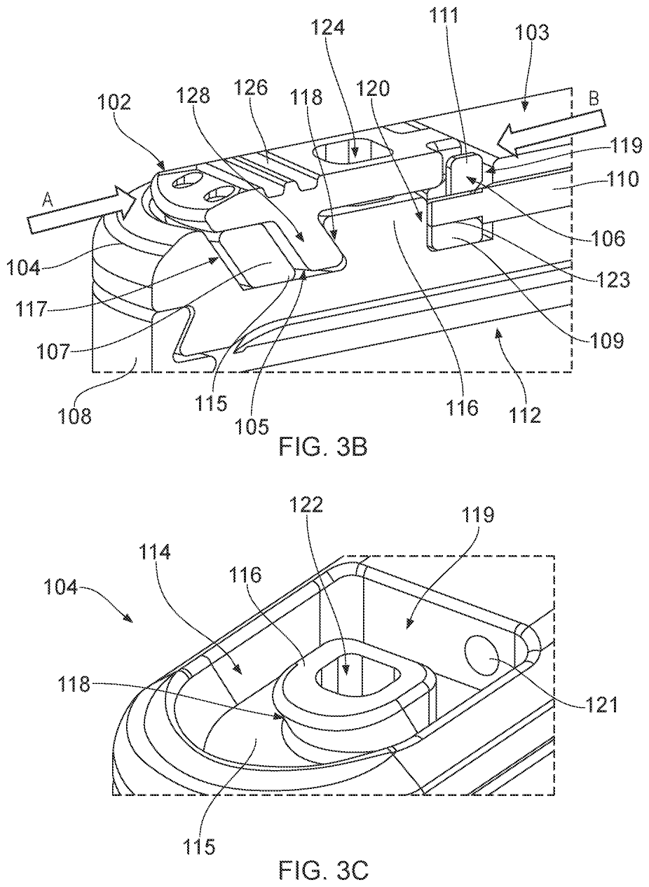

FIGS. 3A-C illustrate a first example of the present disclosure, in which the electrode assembly of an end effector 100 (also referred to herein as the RF tip) of an electrosurgical instrument is provided with an improved retention mechanism. The end effector 100 comprises an active electrode 102 for tissue treatment, also referred to as the “active tip”, an insulating casing 104 arranged to receive the active electrode 102, and a return electrode 108. The insulating casing 104 is provided between the active electrode 102 and the return electrode 108 to physically separate those components, and may be formed from any suitable material, such as ceramic. In use, the active electrode 102 and the return electrode 108 receive the RF power signal from the generator 1 (not shown) to deliver treatment to a tissue treatment site. A retainer 106 is also provided for retaining the active electrode 102 within the insulating casing 104, as will be described in more detail below. As will be described in more detail below, the active electrode 102 and the retainer 106 are assembled within the insulating casing 104 and welded together such that they are mechanically and electrically connected. An electrical conductor 110 also extends along a channel 121 within the insulating casing 104 and into the retainer 106, with the electrical conductor 110 extending down along the shaft of the instrument 3 and back to the handpiece 12 for connection to the generator 1, to thereby deliver the RF power signal to the active electrode 102. On the opposing side (partially shown in FIGS. 3A-B), a lumen 112 is provided within the return electrode 108 that extends down through the shaft of the instrument 3 to the suction tube 14 (see FIG. 1). The distal end of the lumen 122 also comprises the components of the mechanical shaver, specifically, the rotating shaver blade (such as that shown in FIG. 2B).

To receive and retain the active tip 102, the insulating casing 104 comprises a cavity 114, the cavity 114 having a base surface 115 with an interlocking projection 116 protruding therefrom, as shown by FIG. 3C. The interlocking projection 116 is defined by a first retention surface 118 that extends in the direction of the distal end of the insulating casing 104 at an acute angle (for example, between 30° and 65°) relative to the longitudinal axis X of the end effector 100, and a second retention surface 120 that extends in a direction perpendicular to the longitudinal axis X (i.e. at a right angle). The interlocking projection 116 also comprises a suction aperture 122, which provides an opening to the lumen 112 for transporting fluids from tissue treatment site. The distal internal wall 117 of the cavity 114 also extends towards the distal end of the insulating casing 104 at an obtuse angle relative to the longitudinal axis X (for example, between 85° and 150°), such that the distal internal wall 11 of the cavity 114 and the first retention surface 118 of the projection 116 are substantially parallel. Similarly, the proximal internal wall 119 of the cavity 114 extends in a direction perpendicular to the longitudinal axis X, such that the proximal internal wall 119 and the second retention surface 120 of the projection 116 are substantially parallel.

To assemble the RF tip 100, the retainer 106 is inserted to the cavity 114. The retainer 106 is configured to fit around the projection 116 such that it sits against the internal walls of the cavity 114. In this respect, the retainer 106 comprises a distal portion 107 that is configured such that it lies against the distal internal wall 117 of the cavity 114, leaving a space 105 between the distal portion 107 and the interlocking projection 116. Furthermore, the walls of the distal portion 107 are also angled such that they run parallel with the distal internal wall 117 and the first retention surface 118, the outer wall of the distal portion 107 abutting the distal internal wall 117. The retainer 106 is then configured to fill the remainder of the cavity 114, with a proximal portion 109 being configured to fit within the space between the proximal internal wall 119 and the second retention surface 120. The proximal portion 109 also comprises a protruding feature 111 that extends out of the cavity 114 such that it protrudes above the top surface 103 of the insulator 104.

As shown in FIGS. 3A-B, the proximal portion 109 also comprises a cavity 123 for receiving the conductor 110 via the channel 121, for connecting the RF tip 100 to an electrical supply (e.g., the generator 1). In this respect, it will be appreciated that the retainer 106 will be formed of some suitable electrically conductive material.

Once the retainer 106 is in place, the electrode 102 can be inserted such that it sits over the top of the retainer 106 and the interlocking projection 116, thereby protruding above the top surface 103 of the insulator 104. As shown in FIGS. 3A-B, the active electrode 102 comprises a planar tissue treatment surface 126, which may include one or more projections or protrusions to concentrate the electric field at the locations of the projections. The projections may also serve to create a small separation between the planar top surface of the active electrode 102 and the tissue to be treated at the surgical site. This allows conductive fluid to circulate over the planar surface and avoids overheating of the electrode or the tissue. A suction aperture 124 is also provided, which is positioned so as to align with the suction aperture 122 of the insulator 104 once assembled. This therefore forms a path from the suction aperture 124 to the suction pump 10 for transporting fluids from the treatment site.

The active electrode 102 comprises a retention feature 128 that extends down from the lower surface (i.e. the opposite side to the tissue treatment surface 126), which is configured to fit within the space 105 between the distal portion 107 of the retainer 106 and the interlocking projection 116. That is to say, the retention feature 128 is also angled relative to the longitudinal axis X such that the external walls are parallel with the first retention surface 118 and the distal portion 107 of the retention 106. The proximal end of the active electrode 102 is also configured to mate with the protruding feature 111 of the retainer 106.

Once assembled, a first biasing force in the proximal direction (denoted by arrow A in FIG. 3B) may be exerted on the active electrode 102 so as to bias the interlocking feature 128 against the first retention surface 118, and a second biasing force (denoted by arrow B in FIG. 3B) in the distal direction similarly exerted on the retainer 106 (for example, by the means of the protruding feature 111) so as to bias the proximal portion 109 of the retainer 106 against the second retention surface 120. Whilst the biasing forces are being exerted, for example, using a mechanical jig or some other suitable equipment, the active electrode 102 and the retainer 106 are welded together, or joined using some other suitable method, such that they are mechanically and electrically connected.

In other arrangements, the biasing forces may be applied using one or more strong magnets so as to bias the active electrode 102 and retainer 2016 without physically touching them, thereby making it easier to weld or join the components at the same time. For example, a strong magnet (not shown) placed in the centre of the suction apertures 122, 124 would pull both components inwards prior to welding.

The addition of biasing forces during production helps to mitigate any clearances in the interlock mechanism caused by part-to-part variation that may occur during manufacture. As the interlock clearances are removed before the active electrode 102 and retainer 106 are welded together, the resulting interlock with the first and second retention surfaces 118, 120 is as robust as possible for each individual assembly, regardless of the variance in component tolerance. Furthermore, the overall active electrode area is reduced, with no portion of the insulator 104 being exposed within the tissue treatment surface 126, thereby improving the performance of the ablation and coagulation functions.

However, the step of applying external biasing forces may be omitted if the active electrode 102 and the retainer 106 have tight manufacturing tolerances and a relatively tight fit within the cavity 114 of the insulator 104. In this respect, if the part-to-part variation is very low, the same mechanical intent may be achieved without the need to bias the components during the welding process.

Whilst the above example shows the first retention surface 118 being an angled surface and the second retention surface 120 being a vertical surface, it will of course be appreciated that this arrangement may be reversed. That is to say, the first retention surface 118 and the retention feature 128 of the electrode 102 may be vertical, whilst the second retention surface 120 and the proximal portion 109 of the retainer 206 may be angled.

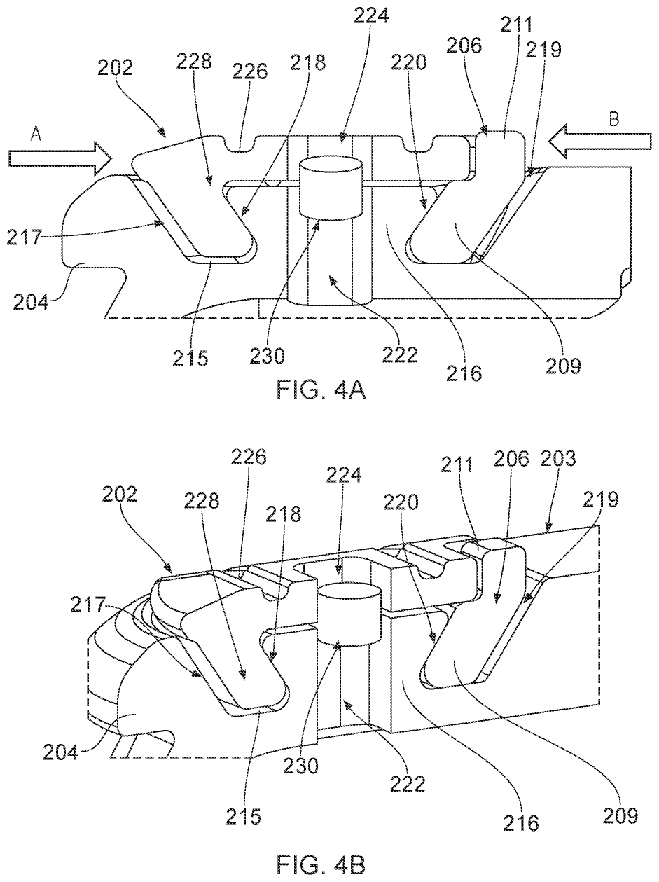

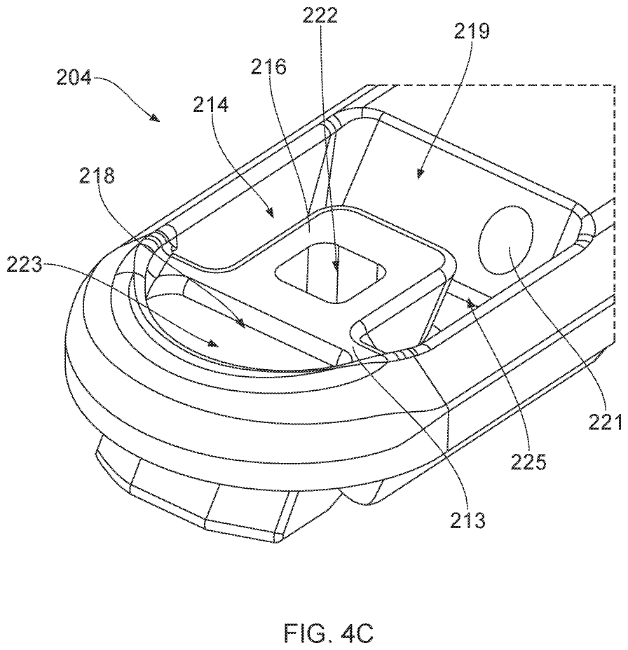

FIGS. 4A-C illustrate a second example in accordance with the present disclosure. As with the first example, the RF tip comprises an active electrode 202, an insulator 204 and a retainer 206. As before, the insulator 204 comprises an interlocking projection 216 protruding from the base surface 215 of an internal cavity 214, with the first retention surface 218 of the interlocking projection 216 extending in a distal direction an acute angle relative to the longitudinal axis of the RF tip. As shown in FIG. 4C, the first retention surface 218 may be that of a wall 213 that extends along the width off the cavity 214 such that it divides the cavity 214 into two sections, a distal cavity 223 being provided at the distal end of the RF tip and a U-shaped proximal cavity 225. As such, the wall 213 connects the interlocking projection 216 to the outer perimeter of the insulator 204 to further increase the mechanical strength of the interlocking projection 216. As with the previous example, the distal internal face 217 of the distal cavity 213 extends at an angle such that it runs parallel with the first retention surface 218 of the interlocking projection 216. By dividing the cavity 214 in this way and anchoring the interlocking projection 216 to the walls of the cavity 214, the distal cavity 223 provides a larger load-bearing interlock area that can withstand larger clinical forces. Similarly, the dividing wall 213 acts to increase the strength of the interlock between the interlocking projection 216 and the active electrode 202. It will however be appreciated that the interlocking projection 216 may be freestanding as it is in FIG. 3C.

In this second example, the second retention surface 220 of the interlocking projection 216 also extends at an acute angle relative to the longitudinal axis of the RF tip, the second retention surface 220 extending in the proximal direction. Likewise, the proximal internal face 219 of the proximal cavity 225 also extends in the proximal direction at an obtuse angle relative to the longitudinal axis, such that the second retention surface 220 and the proximal internal face 219 are parallel with each other. As with the previous examples, the insulator 204 also comprises a suction aperture 222 and a channel 221 for receiving an electrical conductor (not shown) to connect the RF tip to an electrical supply. However, the entry point of the channel 221 must be on provided on an angled plane to correspond to the angled internal wall 219.

In this example, the retainer 206 is configured to be received by the proximal cavity 225 and comprises a proximal interlocking portion 209 that is configured to fit within the space provided between the second retention surface 220 and the proximal internal wall 219. In this respect, the proximal portion 209 is angled such that the walls of the proximal portion 209 sit flush against the second retention surface 220 and the proximal internal wall 219. As with the previous example, the retainer 206 comprises a protruding feature 211 that extends out of the cavity 214 such that it protrudes above the top surface 203 of the insulator 204.

The active electrode 202 is configured in substantially the same way as the active electrode 102 described with reference to the first example, the active electrode 202 comprising a tissue treatment surface 226 that sits above the top surface 203 of the insulator 204, a suction aperture 224, and a retention feature 228 that is configured to be received in the space provided between the first retention surface 218 and the distal internal face 217. In this respect, the external walls of the retention feature 228 are also angled such that they abut both the distal internal face 217 and the first retention surface 218. As before, the active electrode 202 is also configured to mate with the protruding feature 211 of the retainer 206.

Once assembled, a first biasing force may be exerted on the active electrode 202 in the proximal direction (denoted by arrow A in FIG. 3B) so as to bias the interlocking feature 228 against the first retention surface 218, and a second biasing force exerted on the retainer 206 (denoted by arrow B in FIG. 3B) in the distal direction so as to bias the proximal portion 209 of the retainer 206 against the second retention surface 220. As described above, the biasing forces may be exerted by any suitable means such as a mechanical jig, or by placing a strong magnet (230) placed in the centre of the suction apertures 222, 224 to put the electrode 202 and the retainer 206 together. Whilst the biasing forces are being exerted, the active electrode 202 and the retainer 206 are welded together, such that they are mechanically and electrically connected. Once again, these opposing biasing forces can help to mitigate any clearance between the interlocking features, to thereby provide a more robust mechanical retention when the retainer 206 and the active electrode 202 are welded, regardless of the variance in component tolerance. However, in this example, the dual interlocking features can obviate the need to apply biasing forces during the welding process, particularly if there is low part-to-part variation during manufacture.

As in the first example, the overall active electrode area is reduced, with no portion of the insulator 204 being exposed within the tissue treatment surface 226, thereby improving the performance of the ablation and coagulation functions. In the second example, however, the retainer 206 has been shortened so it no longer reaches under the retention feature 228 of the electrode 202. This allows additional space within the cavity 214 to include a second angled interlock between the retainer 206 and the second retention face 220 of the insulator 204, further strengthening the mechanical retention of the welded active electrode 202 and retainer 206 within the insulator 204.

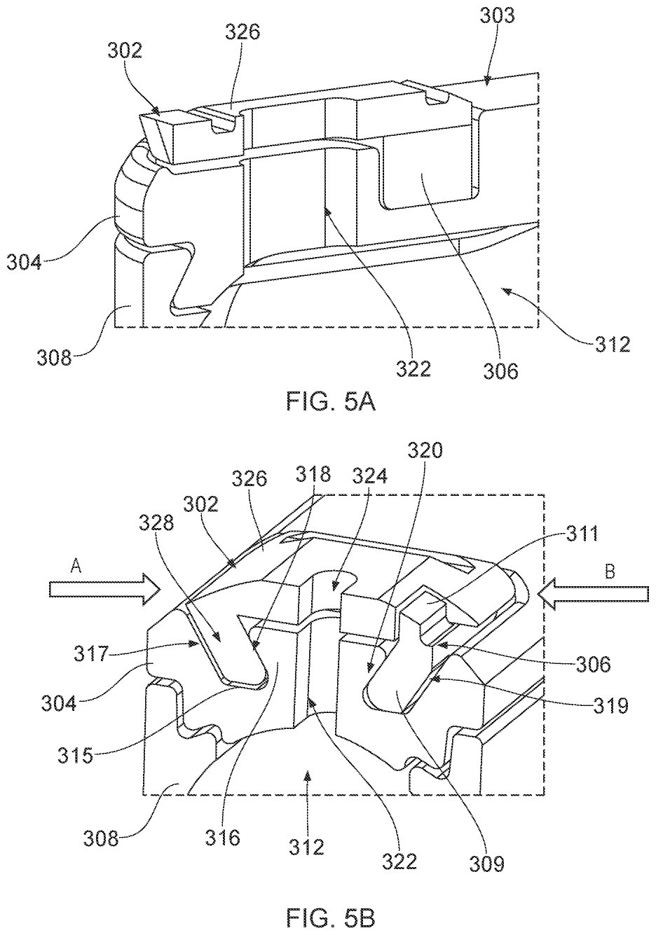

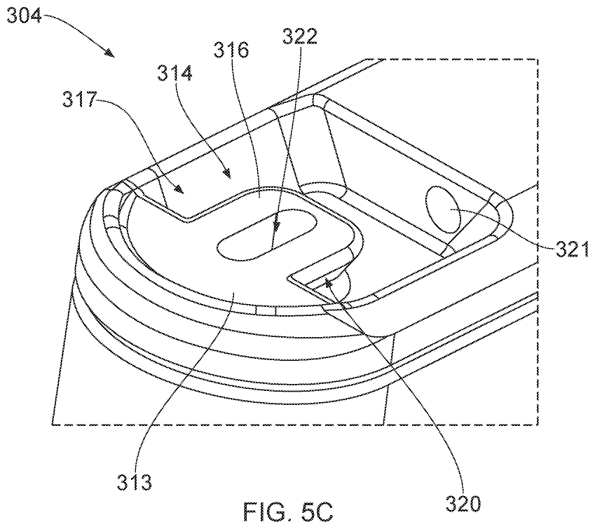

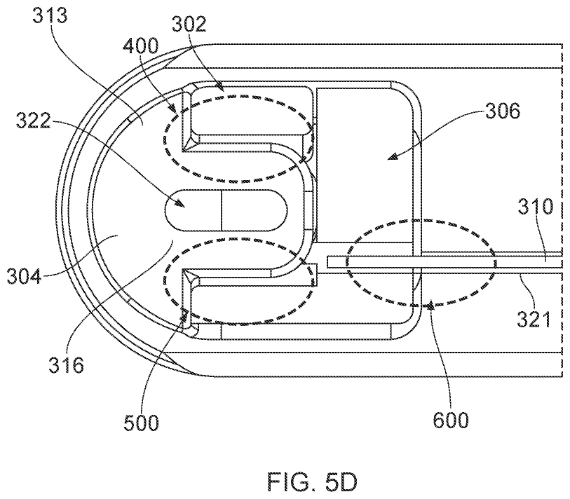

FIGS. 5A-D illustrate a third example of the present disclosure. The third example is similar to the first example, however, the active electrode 302 and retainer 306 arrangement is effectively rotated 90° about an axis perpendicular to the longitudinal axis of the RF tip. As such, the interlocking projection 316 protruding from the base surface 315 of the insulator cavity is also rotated by a corresponding 90°, such that the first and second retention surfaces 318 and 320 extend in opposing lateral directions perpendicular to the longitudinal axis of the RF tip. It is thus the side internal walls 317 and 319 that are configured to run parallel to the first and second retention surfaces 318 and 320 respectively. As such, the cavity 314 in the insulator 304 has a substantially U-shaped configuration around the interlocking projection 316, with the interlocking projection 316 again being connected to the outer perimeter of the insulator 304 via a wall 313.

The retainer 306 and the active electrode 302 have a similar configuration to that shown in FIGS. 4A-C, with the retention feature 328 of the active electrode 302 being received in the space between the first side wall 317 and the first retention surface 318 of the interlocking projection 318, and retainer 306 have a substantially L-shaped configuration (see FIG. 5D) with the interlocking feature 309 being received in the space between the second side wall 319 and the second retention surface 320 of the interlocking projection 318. As such, during the welding process, the biasing forces (denoted by arrows A and B) may be exerted in a lateral direction perpendicular to the longitudinal axis using any suitable technique as described above. In this respect, FIG. 5D illustrates the three areas (400, 500 and 600) of mechanical retention that prevents the active electrode 302 from lifting away from the insulator 304. The first two areas 400 and 500 are provided by the two undercuts between the retention surfaces 318, 320 of the interlocking projection 316 and the active electrode 302 and the retainer 306, whilst the third area 600 is provided by the electrical conductor 310 connection with the retainer 306 via the internal channel 321.

As such, this arrangement provides the same benefits described with reference to the first and second examples, with the additional advantage of that the design of the interlocking projection 316 is not linked to the entry to the channel 321 in the proximal internal wall of the cavity 314. Therefore, a double interlock is achieved to aid tip retention robustness, whilst improving the ease with which the insulator 304 is manufactured, since the conductor 310 enters the cavity 314 on a vertical face, which vastly reduces the likelihood of excess material building up around the entry port during moulding. Furthermore, this provides three distributed anchor points (400, 500 and 600) to secure the active electrode 302 within the insulator 304, further improving the strength of the mechanical retention.

Various further modifications to the above described embodiments, whether by way of addition, deletion or substitution, will be apparent to the skilled person to provide additional embodiments, any and all of which are intended to be encompassed by the appended claims.

The electrosurgical instrument and/or end effector disclosed herein can be designed to be disposed of after a single use, or they can be designed to be used multiple times. In either case, however, the electrosurgical instrument and/or end effector can be reconditioned for reuse after at least one use. Reconditioning can include a combination of the steps of disassembly of the electrosurgical instrument, followed by cleaning or replacement of particular pieces, and subsequent reassembly. In particular, the electrosurgical instrument can be disassembled, and any number of particular pieces or parts of the device (such as the end effector) can be selectively replaced or removed in any combination. Upon cleaning and/or replacement of particular parts, the electrosurgical instrument can be reassembled for subsequent use either at a reconditioning facility, or by a surgical team immediately prior to a surgical procedure. Those of ordinary skill in the art will appreciate that the reconditioning of an electrosurgical instrument can utilize a variety of different techniques for disassembly, cleaning/replacement, and reassembly. Use of such techniques, and the resulting reconditioned electrosurgical instrument, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The electrosurgical instrument may also be sterilized using any other technique known in the art, including but limited to beta or gamma radiation, ethylene oxide, or steam.

1. A method of manufacturing an electrode assembly for use in an end effector of an electrosurgical instrument, comprising:
providing an outer casing comprising an insulating material, wherein the outer casing comprises a cavity, the cavity comprising a base surface, and a first projection extending from the base surface, the first projection comprising a first retention surface and a second retention surface, wherein at least one of the first and second retention surfaces extends away from the base surface at a non-transverse angle relative to a longitudinal axis of the outer casing;
inserting a retainer into the cavity via a first opening in a first wall of the outer casing, wherein the retainer comprises a first retention feature configured to engage the first retention surface;
inserting an electrode into the cavity via the first opening, wherein the electrode comprises a second retention feature configured to engage the second retention surface; and
joining the electrode and the retainer to form the electrode assembly, such that the electrode assembly is mechanically retained within the outer casing.

2. A method according to claim 1, applying a first biasing force on the retainer such that first retention feature is biased against the first retention surface, and a second biasing force on the electrode such that the second retention feature is based against the second retention surface.

3. A method according to claim 1, wherein the retainer further comprises a protrusion configured to extend out of the first opening such that the protrusion extends beyond the first wall, wherein the method optionally further comprises applying a first biasing force to the protrusion.

4. A method according to claim 1, wherein at least one portion of the electrode is configured to extend out of the first opening such that the at least one portion extends beyond the first wall, wherein the method optionally further comprises applying a second biasing force to the at least one portion.

5. A method according to claim 2, wherein the first and second biasing forces are applied by placing a magnet in the vicinity of the electrode and the retainer.

6. A method according to claim 1, wherein the first retention surface extends away from the base surface at an angle transverse to the longitudinal axis, and the second retention surface extends away from the base surface in a direction towards the distal end of the outer casing at a non-transverse angle relative to a longitudinal axis.

7. A method according to claim 1, wherein the first retention surface extends away from the base surface in a direction towards the proximal end of the outer casing at a non-transverse angle relative to a longitudinal axis, and the second retention surface extends away from the base surface in a direction towards the distal end of the outer casing at a non-transverse angle relative to a longitudinal axis.

8. A method according to claim 1, wherein the first retention surface extends away from the base surface in a first lateral direction at a non-transverse angle relative to a longitudinal axis, and the second retention surface extends away from the base surface in a second lateral direction at a non-transverse angle relative to a longitudinal axis.

9. A method according to claim 1, wherein the electrode comprises a tissue treatment surface, the tissue treatment surface being configured to cover the first projection.

10. A method according to claim 9, wherein the first projection comprises a first suction aperture, and the tissue treatment surface comprises a second suction aperture, the electrode being inserted to the cavity such that the second suction aperture aligns with first suction aperture.

11. A method according to claim 1, wherein the first projection is connected to an internal wall of the cavity via an interconnecting wall.

12. A method according to claim 1, wherein the retainer comprises an electrically conductive material.

13. A method according to claim 1, wherein the insulating material comprises a ceramic material.

14. A method according to claim 1, wherein joining the electrode and the retainer comprises welding the electrode and the retainer.

15. A method according to claim 1, wherein the method further comprises providing a conductor, the conductor being received within the cavity via a second opening in an internal wall, wherein the conductor is in electrical communication with the electrode assembly.

16. An electrode assembly for use in an end effector of an electrosurgical instrument, the electrode assembly comprising:
an electrode;
a retainer; and
an outer casing comprising an insulating material, wherein the outer casing comprises a cavity configured to receive the electrode and the retainer, the cavity comprising a base surface, and a first projection extending from the base surface, the first projection comprising a first retention surface and a second retention surface, wherein at least one of the first and second retention surfaces extends away from the base surface at a non-transverse angle relative to a longitudinal axis of the outer casing;
characterised in that the electrode and the retainer are provided in the outer casing by:
inserting a retainer into the cavity via a first opening in a first wall of the outer casing, wherein the retainer comprises a first retention feature configured to engage the first retention surface,
inserting the electrode into the cavity via the first opening, wherein the electrode comprises a second retention feature configured to engage the second retention surface, and
joining the electrode and the retainer to form the electrode assembly, such that the electrode assembly are mechanically retained within the outer casing.

17. An electrode assembly according to claim 16, wherein a first biasing force is applied to the retainer such that first retention feature is biased against the first retention surface, and a second biasing force is applied to the electrode such that the second retention feature is based against the second retention surface.

18. An electrode assembly for use in an end effector of an electrosurgical instrument, the electrode assembly comprising:
an outer casing comprising an insulating material, wherein the outer casing comprises a cavity, the cavity comprising a base surface, and a first projection extending from the base surface, the first projection comprising a first retention surface and a second retention surface, wherein the first and second retention surfaces extend away from the base surface at a non-transverse angle relative to a longitudinal axis of the outer casing; and
an electrode arrangement received in the cavity, the electrode arrangement comprising a retainer and an electrode in electrical communication with the retainer;
wherein the retainer comprises a first retention feature configured to engage the first retention surface, and the electrode comprises a second retention feature configured to engage the second retention surface, such that electrode arrangement is mechanically retained within the outer casing.

19. An electrode assembly according to claim 18, wherein the first retention surface extends away from the base surface in a direction towards the proximal end of the outer casing at a non-transverse angle relative to a longitudinal axis, and the second retention surface extends away from the base surface in a direction towards the distal end of the outer casing at a non-transverse angle relative to a longitudinal axis.

20. An electrode assembly according to claim 18, wherein the first retention surface extends away from the base surface in a first lateral direction at a non-transverse angle relative to a longitudinal axis, and the second retention surface extends away from the base surface in a second lateral direction at a non-transverse angle relative to a longitudinal axis.
